(19) United States
(12) Patent Application Publication
(10) Pub. No.: US 2015/0305709 A1
(43) Pub. Date: Oct. 29, 2015

(54) CONDUCTIVE MEDIUM PAD FOR ULTRASOUND PROBE

(71) Applicant: SONOFAST, INC., Atlanta, GA (US)

(72) Inventors: Keller Anton Tomassi, Atlanta, GA (US); Richard Terry, Atlanta, GA (US); Jorge David Mena, Marietta, GA (US); Gabriela Isabel Lamas, Acworth, GA (US); Stephanie Marie Camstra, The Woodlands, TX (US)

(73) Assignee: SONOFAST, INC., Atlanta, GA (US)

(21) Appl. No.: 14/695,322

(22) Filed: Apr. 24, 2015

Related U.S. Application Data
(60) Provisional application No. 61/983,598, filed on Apr. 24, 2014.
(60) Provisional application No. 62/142,718, filed on Apr. 3, 2015.

(51) Int. Cl. A61B 8/00; B32B 37/12; B29C 39/02

(52) U.S. Cl. CPC A61B 8/4281; B29C 39/025; B32B 37/12; B29K 2033/08

(57) ABSTRACT

The present disclosure discusses conductive pads that are disposable, shape-stable lubricious hydrogel couplants that can be used in place of liquid gel couplants the field of ultrasound. The conductive pad can include dual-surface acoustic hydrogel couplants that have one adhesive surface to provide a position-stable coupling to a transducer or other contact surface and a second surface that is lubricious, enabling it to slide across the surface of a test subject. The lubricious surface of the hydrogel enables low friction repositioning of the transducer. The conductive pad can also include a plurality of layers, each of which can include different properties.

### CROSS-REFERENCE TO RELATED PATENT APPLICATIONS

This application claims priority to U.S. Provisional Patent Application No. 61/983,598 filed on Apr. 24, 2014 and titled “Conductive Medium Pad For Ultrasound Probe” and U.S. Provisional Patent Application No. 62/142,718 and titled “Dual-Surface Hydrogels for Acoustic Coupling” filed on Apr. 3, 2015, both of which are herein incorporated by reference in their entirety.

### BACKGROUND OF THE DISCLOSURE

Ultrasonic energy can be poorly transmitted through air. Air often fills the voids and irregularities between two adjacent surfaces, and can increase the impedance of the ultrasonic energy's transmission through the interface between the adjacent surfaces. Liquid gels have been used to fill the voids, but the liquid gels are messy, and necessitate cleaning of both the test subject and ultrasound transducer after each use. In many procedures the liquid gels must be frequently replenished during the procedure, often from a large container reservoir where sterility is not maintained. Air bubbles can also be formed within the liquid gel because of cavitation by the ultrasonic energy, which can reduce the transmission of the ultrasonic energy between the adjacent surfaces.

### SUMMARY

According to one aspect of the disclosure, an ultrasound conductive pad includes a first lubricious surface, a second, less lubricious surface, and a first hydrogel membrane. The first hydrogel membrane includes one or more water-soluble polymers that are entrapped in an insoluble hydrogel network formed by polymerizing and crosslinking one or more monomers in a formulation with the water-soluble polymer.

In some implementations, the second tacky surface is configured to reversibly couple with at least one of an ultrasound probe and a lithotripsy bellow. The conductive pad can also include a second hydrogel membrane. The first hydrogel membrane can include the first lubricious surface and the second hydrogel membrane can include the second tacky surface.

In some implementations, the first hydrogel membrane includes the one or more water-soluble polymers, and the second hydrogel membrane does not include the one or more water-soluble polymers.

In some implementations, the second tacky surface includes an adhesive surface coupled to the first hydrogel membrane. In some implementations, the one or more water-soluble polymers can include polyvinylpyrrolidone, polyethylene glycol, and polyethylene oxide. The crosslinked hydrogel network can include a multifunctional acrylate. In some implementations, the first hydrogel membrane includes 2-hydroxyethyl methacrylate that is crosslinked with between about 0.5 and about 10.0% multifunctional acrylate based on the total weight of 2-hydroxyethyl methacrylate and the multifunctional acrylate. The multifunctional acrylate can include at least one of a polyethylene glycol dimethacrylate and a polyethylene glycol diacrylate.

According to another aspect of the disclosure, a method of reducing impedance in an ultrasound recording includes providing a conductive pad. The conductive pad includes a first lubricious surface, second tacky surface, and a first hydrogel membrane. The first hydrogel membrane includes 2-hydroxyethyl methacrylate that is crosslinked with between about 0.5% and about 10.0% multifunctional acrylate based on the total weight of 2-hydroxyethyl methacrylate and the multifunctional acrylate, and includes one or more water-soluble polymers. The one or more hydrophilic polymers are entrapped within the crosslinked hydrogel network. The method also includes coupling the conductive pad to a transducer, and transmitting ultrasonic energy through the conductive pad and into a first surface.

In some implementations, the second tacky surface is configured to reversibly couple with the transducer. The transducer can be a transducer of at least one of an ultrasound probe and a lithotripsy bellow.

In some implementations, the conductive pad also includes a second hydrogel membrane. The first hydrogel membrane can include the first lubricious surface and the second hydrogel membrane can include the second tacky surface. The first hydrogel membrane includes the one or more water-soluble polymers, and the second hydrogel membrane does not include the one or more water-soluble polymers. In some implementations, the second tacky surface can include an adhesive surface coupled to the first hydrogel membrane.

In some implementations, the one or more water-soluble polymers include at least one of polyvinylpyrrolidone, polyethylene glycol, and polyethylene oxide, and the crosslinked hydrogel network is composed of 2-hydroxyethyl methacrylate that is crosslinked with between about 0.5 and about 10.0% multifunctional acrylate based on the total weight of 2-hydroxyethyl methacrylate and the multifunctional acrylate. In some implementations, the hydrogel membrane includes between about 0.5 and about 10.0% multifunctional acrylate based on the total weight of 2-hydroxyethyl methacrylate and the multifunctional acrylate.

According to another aspect of the disclosure, a method of manufacturing a conductive pad includes preparing a first formulation. The first formulation includes one or more water-soluble polymers dissolved in water. The method also includes preparing a second formulation that includes a water-soluble monomer and a crosslinking agent. The first and second formulations are combined to form a third formulation. The third formulation is cast into a casting mold, and then cured. In some implementations, the method includes combining a water-based catalyst to the third formulation.

In some implementations, a surface of the casting mold has a water contact angle greater than about 90°. The method can also include forming a first surface at an interface of the surface of the casting mold and the cast third formulation, and forming a second surface at an interface of the cast third formulation and a gas. In some implementations, the first surface has a greater lubricity than the second surface.

In some implementations, the method also includes forming a fourth formulation that does not include a water-soluble polymer to produce a tacky surface on the conductive pad. In some implementations, the fourth formulation is cast into the casting mold and at least partially cured, and then the third formulation is cast on top of it to produce one lubricious surface and the two layers are cured together. In some implementations, the third formulation is cast first and partially cured, and then the fourth formulation is cast on top of it and the two layers are cured together.

In some implementations, the method includes casting the third formulation onto an adhesive backing material before it is cured. In some implementations, an adhesive backing with good adhesion properties to the third formulation is first prepared by adhering a fibrous material to a pressure sensitive adhesive, and then casting the third formulation on top of the fibrous side before curing. In some implementations, the one or more water-soluble polymers include at least one of polyvinylpyrrolidone, polyethylene glycol, and polyethylene oxide, and the water-soluble monomer comprises at least one hydrophilic ethylenically-unsaturated monomer, and the crosslinking agent comprises a multi-olefinic crosslinking agent.

In some implementations, the water-soluble monomer includes 2-hydroxyethyl methacrylate and the crosslinking agent includes a multifunctional acrylate. In some implementations, the second formulation includes between about 0.5% and about 10.0% multifunctional acrylate based on the total weight of 2-hydroxyethyl methacrylate and the multifunctional acrylate. In some implementations, the multifunctional acrylate includes at least one of a polyethylene glycol dimethacrylate and a polyethylene glycol diacrylate.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 illustrates an example system for using an ultrasound system with a conductive pad.

FIG. 2A-2C illustrate example conductive pads for use with the system illustrated in FIG. 1.

FIG. 3 illustrates a flow chart of an example method for manufacturing a conductive pad for use with the system illustrated in FIG. 1.

FIG. 4 illustrates a flow chart of an example method for reducing impedance in an ultrasound recording using the system illustrated in FIG. 1.

### DETAILED DESCRIPTION

The various concepts introduced above and discussed in greater detail below may be implemented in any of numerous ways, as the described concepts are not limited to any particular chemistry or manner of implementation. Examples of specific implementations and applications are provided primarily for illustrative purposes.

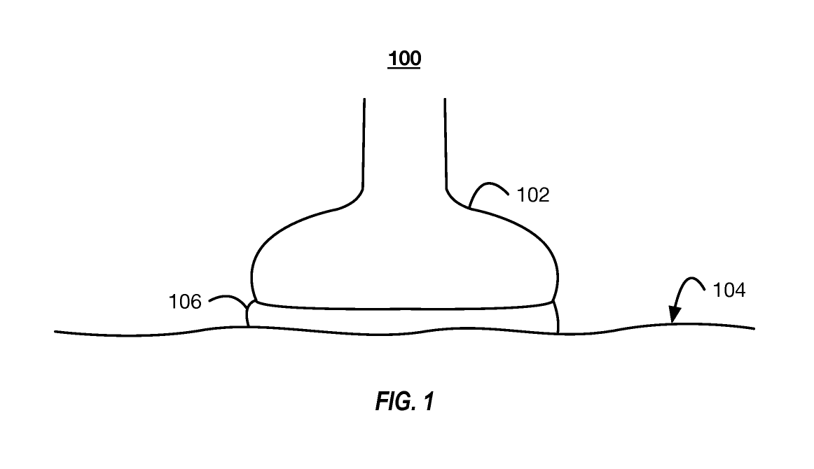

FIG. 1 illustrates an example system 100 for using an ultrasound system with a conductive pad. The system 100 includes a probe 102 that is used to deliver ultrasonic energy below a surface 104. The system 100 includes a conductive pad 106 that is configured to acoustically couple the probe 102 to the surface 104. In some implementations, the ultrasonic energy is used to image below the surface 104 (e.g., to create an ultrasound image) or for the destruction of hardened masses (e.g., the destruction of kidney stones with a lithotripsy device).

The probe 102 of system 100 can be any type of probe that emits ultrasonic energy. For example, the probe 102 can be the probe of an ultrasound machine. In another implementations, the probe 102 can be a lithotripsy bellow. In other implementations, the probe 102 can be any other type of medical or non-medical ultrasound transducer.

The surface 104 can be the skin of a patient. For example, when the probe 102 is the probe of an ultrasound machine, the probe 102 can be used to image below the surface of the patient's skin. The surface 104 may be the skin of the patient above the target location of the ultrasound imaging.

The pad 106 of the system 100 is configured to acoustically couple the probe 102 to the surface 104. The conductive pad 106 and the method of manufacturing the conductive pad 106 is described in further detail below, but in general the conductive pad 106 is configured to produce intimate contact between the surface of the probe 102 that emits the ultrasonic energy and the surface 104. The intimate contact between the surface of the probe 102 and the surface 104 can reduce the impedance caused when air is present between the surface of the probe 102 and the surface 104.

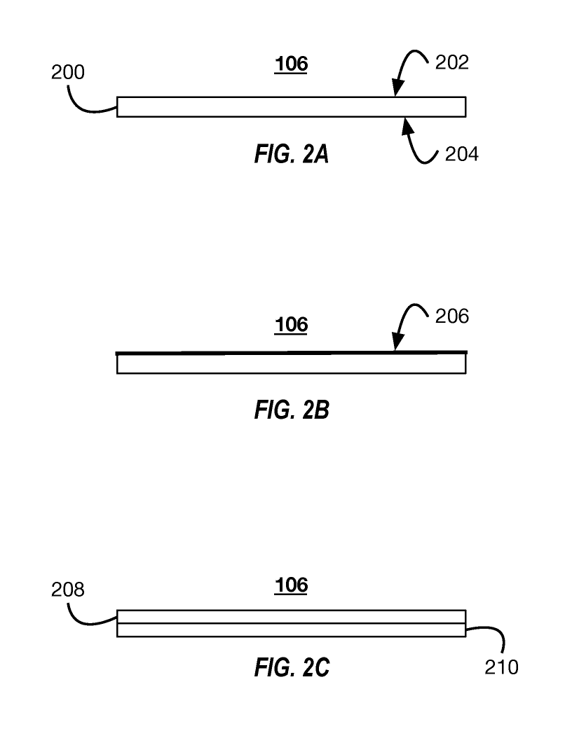

Also referring to FIGS. 2A-2C, in some implementations, the conductive pad 106 is configured as a single layer hydrogel membrane, a single layer hydrogel with an adhesive backing, or a multi-layer hydrogel membrane with dual surfaces. FIG. 2A illustrates a conductive pad 106 with a single layer 200 and a first surface 202 and a second surface 204. In the configurations with dual surfaces, one surface can be a lubricious surface that enables repositionable coupling to the surface 104 (e.g., the lubricious surface creates a low coefficient of friction between the conductive pad 106 and the surface 104). An opposing surface of the conductive pad 106 can be substantially non-lubricious, tacky, or less lubricous than the first surface and enables the conductive pad 106 to be reversibly coupled to the probe 102. For example, the lubricious surface enables the conductive pad 106 to slide across the surface 104, and the tacky or less lubricious surface enables the conductive pad 106 to remain affixed to the probe 102 as the conductive pad 106 is slid across the surface 104. In some implementations, the different properties of the first surface 202 and the second surface 204 are produced as a result of the below described casting process. For example, a highly lubricious side of the membrane can be manufactured at the casting material interface by casting the hydrogel onto a hydrophobic surface that has a water contact angle greater than about 90°. When cast into a mold made of hydrophobic material, a highly lubricious surface 202 is created at the mold interface and a less lubricious surface, 204, is created at the air interface of the conductive pad 106 in FIG. 2A. In some implementations, the casting surface is a high-energy surface such as glass, which has a water contact angle that is less than 60°. The high-energy surface produces a less lubricious surface at the mold interface and highly lubricious surface at the air interface. In some implementations, the hydrogel is cast onto a surface with a water contact angle between 20° and 60°, between 40° and 60°, or between 50° and 60°.

FIG. 2B illustrates a single layer hydrogel 106 with an adhesive backing 206. In some implementations, the side opposite the adhesive backing 206 is lubricious. In some implementations, to better couple the conductive pad 106 to the probe 102, the adhesive backing 206 is coupled to the conductive pad 106. The adhesive backing 206 can include a combination of a fibrous material or fusible web-tape and a double-sided pressure sensitive adhesive.

FIG. 2C illustrates a dual-layer hydrogel 106. The dual-layer hydrogel 106 includes a first layer 208 and a second layer 210. The first layer 208 and the second layer 210 can include different or the same hydrogel polymer composition. For example, the first layer 208, which may be coupled to the probe 102, may be configured to be substantially tackier than the lubricous second layer 210, which interfaces with the patient's skin.

In some implementations, the lubricous side of the conductive pad 106 has a coefficient of friction between about 0.02 and about 0.15, between about 0.02 and about 0.12, or between about 0.02 and about 0.08. In some implementations, the conductive pad 106 of the system 100 is configured to have the same shape as the surface of the probe 102 that emits the ultrasonic energy. The conductive pad 106 can be substantially rectangular, circular, or square in shape. In rectangular or square implementations, the surface area of the pad 106 is between 1 cm² and 20 cm², between 2 cm² and 12 cm², or between 3 cm² and 6 cm². In circular implementations, e.g. for use with lithotripsy, diameter of the conductive pad 106 is between about 50 mm and about 500 mm, between about 70 mm and about 300 mm, or between about 80 mm and about 150 mm. In some implementations, the conductive pad 106 thickness is between about 1 mm and about 25 mm, between about 2 mm and about 20 mm, between about 2 mm and about 15 mm, or between about 2 mm and about 10 mm. In some implementations, the conductive pad 106 is substantially larger than the surface of the probe 102. For example, the conductive pad 106 can be a pad that is laid over the area where the probe 102 will be moved. In this example, the tacky side of the conductive pad 106 may be placed toward the surface 104 and the lubricious surface of the conductive pad 106 toward the probe 102 such that the conductive pad 106 remains in place on the surface 104 as the probe 102 slides across the lubricious surface of the conductive pad 106.

The conductive pad 106 includes water-soluble polymers incorporated into high water content hydrogels. The water-soluble polymers can be incorporated into the hydrogels by dissolving the water-soluble polymers in water and combining with a hydrophilic monomer, crosslinking agent, and catalyst to prepare a pre-cure hydrogel formulation (referred to generically herein as a “formulation”). The crosslinking agent can be a molecule that can polymerize with the hydrogel monomer and propagate two or more polymer chains.

The crosslinking process connects the polymerizing chains into a molecular network that is “crosslinked.” The formulation is catalyzed or “cured” to effect polymerization and crosslinking, producing the shape-stable hydrogel. Once cured, the water-soluble polymer is entrapped in the crosslinked network of the hydrogel structure, and the water-soluble polymer remains solubilized by the high water content of the hydrogel. This structure of the water-soluble polymer within the hydrogel can be referred to as an interpenetrating network (IPN). When high molecular weight water-soluble polymers are used as an IPN in the hydrogels, the high molecular size of the water-soluble polymers can prevent the water-soluble polymers from releasing from the hydrogel surface due to molecular entanglement within the crosslinked hydrogel network.

As described below, a conductive pad 106 with a substantially smooth and slippery surface can be produced by controlling the ratio of water-soluble polymer to hydrogel polymer and the percent water in the formulation. In some implementations, if too much water-soluble polymer is combined with too high of a water content, the resulting hydrogels can be too weak when used alone for use in many ultrasound applications. Conversely, if too low of a ratio of water-soluble polymer to hydrogel polymer is used in combination with a lower amount of water in the hydrogel, the result can be a hydrogel that is very strong and durable but not very slippery.

In some implementations, the water soluble polymer that is entrapped within the hydrogel to produce a slippery surface can include, but is not limited to, polyvinylpyrrolidone (PVP), polyethylene glycol (PEG) and polyethylene oxide (PEO), a high molecular weight version of PEG. In some implementations, the molecular weight (mw) of the polymer ranges from the very low (<1000 Daltons) to the very high (>1 million Daltons). As defined herein, a molecular weight above 100,000 Daltons is classified as a high molecular weight polymer. For example, PVP K-90 has an average molecular weight of about 360,000 and is considered to be a high mw water-soluble polymer. In some implementations, PVP K-90 is a preferred water-soluble polymer. In other implementations, the PVP can have a molecular weight between 2000 and 1,500,000 Daltons.

In some implementations, the polymers used to form the conductive pad 106 can be an acrylate monomer, such as 2-hydroxyethyl methacrylate (HEMA). In some implementations, HEMA can be polymerized by free radical initiation of the acrylate vinyl group to produce a linear HEMA polymer. In some implementations, the monomer is an ethylenically-unsaturated monomer and can include methacrylic acid, salts of methacrylic acid, esters of methacrylic acid, salts and acids of esters of methacrylic acid, amides of methacrylic acid, N-alkyl amides of methacrylic acid, salts and acids of N-alkyl amides of methacrylic acid, N-vinylpyrrolidone, acrylamide, acrylamide derivatives, methacrylamide, methacrylamide derivatives, acrylamide, N-isopropylacrylamide, 2-hydroxyethyl methacrylate (HEMA), 2-hydroxypropyl methacrylate, acrylic acid, 2-acrylamido-2-methyl-1-propanesulfonic acid, 3-sulfopropyl acrylate potassium salt, [2-(acryloyloxy)ethyl]trimethylammonium methyl sulfate and its inorganic salts, [2-(methacryloyloxy)ethyl]trimethylammonium methyl sulfate and its inorganic salts, or a combination thereof. In some implementations, the crosslinking agent includes ethylene glycol dimethacrylate, ethylene glycol diacrylate, poly(ethylene glycol)dimethacrylate, poly(ethylene glycol)diacrylate, poly(ethylene glycol)diacrylamide, N,N′-methylenebisacrylamide, piperazine diacrylamide, glutaraldehyde, epichlorohydrin, crosslinking agents containing 1,2-diol structures, crosslinking agents containing functionalized peptides, and crosslinking agents containing proteins.

In some implementations, the polymer of the conductive pad 106 can be crosslinked with a difunctional or a multi-functional acrylate monomer. A multifunctional acrylate can be a molecule that has two or more polymerizable acrylate groups. For example, a difunctional acrylate monomer can be ethylene glycol dimethylacrylate (EGDM), which can enable an aqueous solution of HEMA to be catalyzed to produce a shape-stable, crosslinked hydrogel network that can contain between about 50% and about 90% water, between about 60% and about 80% water, or between about 65% and about 75% water. In some implementations, the formulation can include between about 0.5% and about 10.0% multifunctional acrylate, between about 1% and about 5% multifunctional acrylate, or between about 1.0 and about 3.0% multifunctional acrylate based on the total weight of 2-hydroxyethyl methacrylate and the multifunctional acrylate. In some implementations, a higher molecular weight acrylate crosslinking agent is used. For example, polyethylene glycol dimethacrylate (PEGDM), where the single ethylene glycol of EGDM is replaced with polyethylene glycol (PEG), can be used as the crosslinking agent to produce the conductive pad 106. The repeating units of ethylene glycol provide a greater distance between the reactive acrylate end groups producing longer crosslinks between the HEMA chains, and this can result in hydrogels with greater strength, durability, and elasticity when compared with the hydrogels made with EGDM. The PEG in PEGDM can contain 8 to 9 repeating units of ethylene glycol. In some implementations, the PEGDM can contain between 2 and 500, between 5 and 100, or between 5 and 25 repeating units of PEG.

In some implementations, the catalyst used to form the conductive pad 106 can polymerize the monomers by free radical initiation. For example, free radicals can attack the carbon-carbon double bond of the acrylate vinyl group, leading to chain growth propagation by reaction with other vinyl groups in the formulation. In some implementations, the catalyst can produce free radicals by either thermal decomposition or photolysis by ultraviolet light. The catalysts can include benzoyl peroxide, ammonium persulfate, sodium bisulfite and potassium persulfate. In some implementations, the catalyst is sodium persulfate (SPS)—an inorganic peroxide. SPS is readily soluble in water, and easily formulated into the water of the HEMA hydrogel formulations. SPS can be activated by thermal decomposition at relatively low temperatures—for example from about 75° C. to 90° C. In some implementations, the potassium salt of persulfate can be used as the catalyst. In some implementations, the catalysts is selected such that the catalyst does not generate a gaseous byproduct, such as oxygen or nitrogen, upon decomposition, because a gaseous byproduct of the reaction could produce microscopic bubbles that might attenuate ultrasound energy passing through the conductive pad 106.

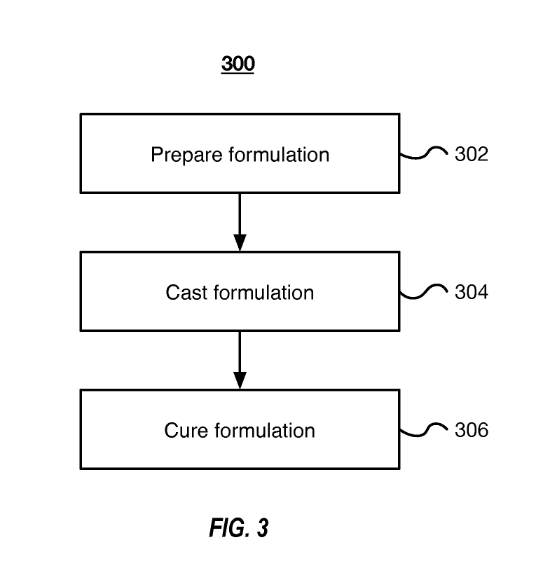

FIG. 3 illustrates a flow chart of an example method 300 for manufacturing a conductive pad. The method 300 can include preparing a formulation (step 302). The formulation is then cast into a mold or onto a surface (step 304), and then the formulation is cured (step 306).

As set forth above, the example method 300 includes preparing the formulation (step 302). Specific example formulations are described below in the Examples Section. In general, preparing the formulation includes preparing a hydrogel solution that includes a monomer or polymer, a crosslinking agent, a catalyst, and a water-soluble polymer. For example, and as further described below, the formulation can include preparing a first solution of water soluble polymer in water, followed by the addition of hydrogel monomer, crosslinker and catalyst to the formulation followed by mixing until the formulation is homogeneous. In one example, a first solution is prepared by dissolving PVP K-90 in distilled water, a second solution of PEGDM crosslinker and HEMA monomer is prepared, and then a third solution of SPS in water is prepared. The above three solutions are then combined to form the formulation.

In some implementations, the conductive pad includes a plurality of layers. The plurality of layers can each include a different formulation. For example, the conductive pad can be manufactured to include a first layer that is lubricous and a second layer that is tacky or adhesive in nature. The use of a plurality of layers can enable the production of conductive pad that has two different surfaces, one for securement to a probe, and the other for low friction repositioning of the probe on the surface of the patient or on the surface of another test object. In one example, a first layer that is configured to be lubricous can be produced as described above, and a second layer configured to be tacky can include the same composition except the water soluble polymer (e.g., PVP) is not be added to the formulation of the second layer.

In some implementations, one or more layers of the conductive pad also include additional chemicals such as, but not limited to, slip additives, humectants to retard drying, salts, emollients, antimicrobial agents to prevent the growth and transmission of pathogenic organisms, and preservative agents to prevent mold growth.

Referring to FIG. 3, once the formulation is prepared, the formulation can be cast into a mold (step 304). Casting the formulation can include pouring the formulation into a mold and then degassing the formulation to remove bubbles and dissolved air within the formulation.

In some implementations, the surface of the casting mold can affect the properties of the formed conductive pad. In some implementations, the surface of the casting mold can be used to generate a dual-surface pad. For example, when the formulation is cast onto a support surface having a high water contact angle, such as a hydrophobic polymer surface, the water-soluble polymer in the formulation can accumulate at the interface with the hydrophobic support surface. The accumulation at the interface of the water-soluble polymer can produce a more lubricious surface on the pad surface facing the casting mold and a less lubricious hydrogel surface at the opposing surface, which shares its interface with air.

When cast onto a material with a lower water contact angle, such as glass, wetting and adhesion of the formulation to the casting surface material can produce a less lubricious surface of the cured hydrogel at the casting surface interface and a more lubricious hydrogel surface at the air interface. Hydrophobic polymers such as polyethylene, polypropylene, polymethylpentene and fluoropolymers all have water contact angles that are greater than 90° and can be used to generate conductive pads that have a lubricious surface at the mold interface and a less lubricious surface at the opposing air interface. More wettable surfaces such as glass have a water contact angles less than 60° and can be used to create conductive pads that can have their most lubricious surface at the air interface and a less lubricious surface at the interface with the low water contact angle material. In some implementations, the casting surface includes glass, glazed ceramic, or a borosilicate glass.

In some implementations, the formulation is cast onto a preformed adhesive sheet. When the formulation is cured, the adhesive sheet can be used to couple the conductive pad to a probe or other transducer. In some implementations, the adhesive is an adhesive hydrogel, and the lubricious hydrogel can bond directly with the adhesive hydrogel by reacting with it or forming an interpenetrating network with its surface before the lubricious hydrogel is cured. In some implementations, the adhesive sheet includes a pressure sensitive adhesive (PSA). Because PSAs can be hydrophobic in nature, the lubricious hydrophilic hydrogels of the invention can adhere poorly to the PSAs, resulting in delamination of the two layers. To enable an improved coupling between the hydrogel and a hydrophobic adhesive, a fibrous layer can be included between the adhesive sheet and the formulation to provide a scaffolding on which the hydrogel can adhere. For example, an adhesive hydrogel sheet is fabricated and a fibrous sheet is bonded to it. The liquid hydrogel formulation is then cast on top of the fibrous layer attached to the adhesive hydrogel and the hydrogel bonds to the fibrous layer when cured. In another example, the formulation can be cast onto the fibrous portion of Velcro® in the bottom of a mold. In this way the liquid hydrogel formulation can infuse within the Velcro fibers before curing. Once cured, the hydrogel material polymerizes around the fibers, locking the hydrogel layer in place onto the hydrophobic adhesive sheet. In some implementations, a sheet of PSA is first adhered to a sheet of fibrous scaffolding material and the formulation is then cast onto the fibrous sheet that is adhered to the PSA. In some implementations, the PSA can be based on, but is not limited to, acrylics, butyl rubbers, natural rubber, nitrile rubber, ethylene-vinyl acetate, silicone rubbers, and styrene-rubber block copolymers.

Referring to the method 300 illustrated in FIG. 3, the cast formulation is then cured (step 306). In some implementations, the cast formulation may be cured by heat, ultraviolet light, or a combination thereof. For example, the cast formulation may be cured by heat for between about 1 hr and about 4 hr or between about 1.5 hr and about 3 hr at between about 170° F. and about 190° F., or between about 175° F. and about 185° F. In some implementations, the cast formulation can be cured in stages. For example, when the conductive pad includes a plurality of layers, a first layer can be partially cured and then a second formulation can be cast over the partially cured formulation and then cured. Furthering this example, a first layer of an adhesive hydrogel formulation can be cast into a mold and partially cured. Next, a lubricious hydrogel formulation can be cast on top of the partially cured adhesive hydrogel, which is then fully cured to form the multiple layer conductive pad. In another example, the lubricious hydrogel can be cast first, partially cured, and then the adhesive hydrogel can be cast on top of the lubricous hydrogel before completing the final cure.

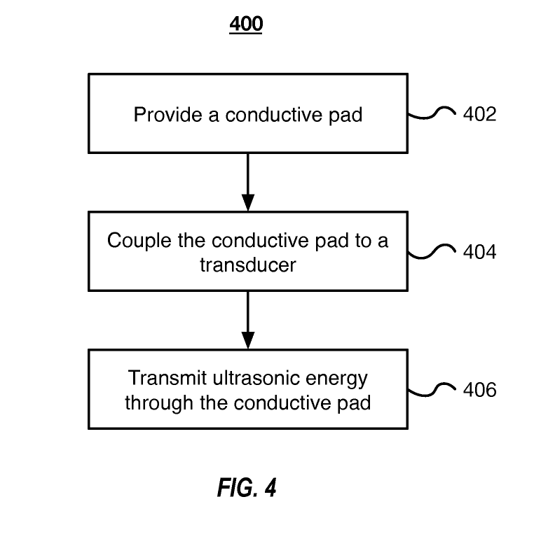

FIG. 4 illustrates a flow chart of an example method 400 for reducing impedance in an ultrasound recording. The method includes providing a conductive pad comprising (step 402). The conductive pad is then coupled to a probe (step 404), and ultrasonic energy is passed through the conductive pad and into a first surface (step 406).

As set forth above, the method 400 can include providing a conductive pad (step 402). The conductive pad can be any of the conductive pads described herein. In some implementations, the conductive pad includes a first lubricious surface and a second tacky surface. The conductive pad can be formulated from one or more water-soluble polymers that are entrapped within a crosslinked hydrogel network. In some implementations, the conductive pad includes a plurality of hydrogel membrane layers. For example, the first lubricious surface can be a surface of a first hydrogel membrane that includes a water soluble polymer and the second tacky surface can be a surface of a second hydrogel membrane that does not include the water soluble polymer.

The method 400 can also include coupling the conductive pad to a transducer (step 404). Also referring to FIG. 1, in some implementations, the traducer is the probe 102 of an ultrasound device or a lithotripsy device. In some implementations, the second tacky surface of the conductive pad is positioned against and coupled to the transducer. The conductive pad can be reversibly coupled to the transducer. For example, a new conductive pad can be applied to an ultrasound-imaging device prior to imaging a patient. After the imaging procedure is completed, the conductive pad can be removed from the transducer and discarded.

The method 400 can include transmitting ultrasonic energy through the conductive pad (step 406). The conductive pad can act as an acoustic couplant between the transducer and the surface into which the ultrasonic energy is transmitted. As a couplant, the conductive pad can reduce the impedance of the ultrasonic energy's transmission into the surface. The conductive pad can increase the efficacy of the ultrasonic energy's transmission by reducing the cavitation that can occur in liquid coupling gels when the ultrasonic energy is transmitted without the conductive pad.

### EXAMPLES

The examples below are embodiments of the lubricious, dual-surface, and dual-layer hydrogels described herein. The examples may be embodied in other specific forms without departing from the spirit or essential characteristics thereof. The forgoing examples are therefore to be considered in all respects illustrative, rather than limiting of the disclosure. Unless otherwise stated, all percentages discussed are weight percent.

### Example 1

### Preparation of a Lubricious Acoustic Coupling Hydrogel

A liquid hydrogel formulation containing PVP was prepared by combining 79.0 g of a 15% by weight solution of PVP K-90 in water with 20.9 g of a solution of 2.8% by weight of PEGDM dissolved in HEMA monomer. The formulation was stirred until homogeneous, and then 3.6 g of a 2.0% solution of sodium persulfate (SPS) in water was added to the formulation. The resulting hydrogel formulation contained 68.3% water, the PVP constituted 36.2% of the polymer solids, and the SPS constituted 0.3% of the hydrogel solids of the formulation. The liquid formulation was cast into a two-sided, rectangular polypropylene mold and degassed under vacuum to remove dissolved air. The mold was then placed in an oven at 175° F. for 3 hours to polymerize the hydrogel into a shape-stable solid. The resulting hydrogel film was removed from the mold and rinsed in distilled water. Both sides of the hydrogel film were highly lubricious and slippery to the touch. When the film was rubbed on the skin, very little residue other than water was left on the skin.

### Example 2

### Fabrication of a Dual-Surface Hydrogel Pad Using a Hydrophobic Polymer Mold

The hydrogel formulation from Example 1 was used, and 34.0 g of the formulation was cast into a 100 mm diameter Petri dish made of polymethylpentene (PMP). The liquid was then degassed under vacuum. A lid was placed on the dish, and the hydrogel was cured in an oven at 175° F. for 2.5 hours. The Petri dish lid was then removed, and the mold was cured for an additional 30 minutes at 175° F. to dry the surface of the hydrogel. The resulting 100 mm diameter hydrogel pad was approximately 4 mm thick. It was dry and slightly tacky on the surface that was open to air, but was highly lubricious on the surface that was in contact with the mold, even after rinsing in distilled water.

### Example 3

### Fabrication of a Dual-Surface Hydrogel Pad Using a Glass Mold

The hydrogel formulation from Example 1 was used, and 24.0 g of the formulation was cast into a 90 mm diameter Petri dish made of borosilicate glass, followed by degassing of the liquid under vacuum. A lid was placed on the glass petri dish and the hydrogel was cured for 3 hours in an oven at 175° F. The resulting 90 mm diameter hydrogel pad was approximately 3 mm thick. It was slightly tacky on the surface that was in contact with the glass, but the surface exposed to air was wet and highly lubricious, even after rinsing in distilled water.

### Example 4

### Fabrication of a Dual-Layer Hydrogel Pad

A tacky hydrogel formulation similar to that described in Example 1 but without PVP was made by first preparing a solution of 1.5% PEGDM in HEMA monomer. The crosslinking level was lowered from 2.8% PEGDM to 1.5% for the layer without PVP because it was found to swell less without PVP in the formulation, and it is desirable to match swelling behavior of the two layers. 7.5 g of this HEMA solution was then combined with 20.9 g of distilled water to produce a clear solution, and then 1.3 g of an aqueous 2% SPS solution was added as a catalyst. This hydrogel formulation without PVP contained 74.7% water and the SPS constituted 0.3% of the total solids of the formulation. 10.0 g of the clear liquid hydrogel formulation was added to a 90 mm glass Petri dish and degassed under vacuum to remove dissolved air. The mold was then covered and placed in an oven at 175° F. for 45 minutes to partially cure the hydrogel, followed by an additional 15 minutes in the oven with the mold open to dry the hydrogel surface. A lubricious hydrogel formulation similar to that prepared in Example 1 was degassed separately under vacuum, and then 22.0 g of the degassed liquid was cast on top of the partially cured hydrogel without PVP. The mold was covered and placed back in the oven at 175° F. for 2 hours to complete the cure of the layered hydrogel. The resulting hydrogel pad was removed from the mold and rinsed in distilled water. It was approximately 4 mm thick, and had one surface that was highly lubricious and an opposing surface that was a tacky adhesive hydrogel.

### Example 5

### Fabrication of a Dual-Layer Hydrogel Pad

A dual-layer hydrogel pad was prepared as described in Example 4 except that a 100 mm PMP Petri dish mold was used. For this larger mold, 11.2 g of tacky hydrogel formulation was added first to the mold, and 24.0 g of lubricious hydrogel formulation was added on top of the partially cured tacky hydrogel. A larger dual-layer hydrogel pad approximately 4 mm thick, with one tacky and one lubricious surface, was produced by this method.

### Example 6

### Fabrication of a Dual-Layer Hydrogel Pad

A dual-layer hydrogel pad was prepared as described in Example 4 except that the lubricious hydrogel formulation was cast first, and either a glass or PMP Petri dish mold was used. Dual-surface hydrogel pads having one tacky and one lubricious surface were produced by this method.

### Example 7

### Fabrication of a Dual-Layer Hydrogel Pad by Coating

A lubricious hydrogel membrane was prepared as described in either Example 2 or Example 3 with only 2 hours total cure time at 175° F. The hydrogel pad was removed from the mold, and placed tacky side up on a clean surface. The tacky surface was allowed to air dry for 30 minutes and then a thin layer of tacky hydrogel formulation from Example #4 was brush-coated onto the dry hydrogel surface. The coated hydrogel pad was then cured for an additional hour in an oven at 175° F.

### Example 8

### Fabrication of a Dual-Layer Hydrogel Pad by Coating

The dual-layer hydrogel membrane as prepared in Example 7 except that the tacky hydrogel layer is cured by a radiant heat source directed only at the tacky hydrogel layer.

### Example 9

### Lubricious Hydrogel with Velcro® Adhesive Backing

A rectangular polypropylene mold was modified by adhering the fibrous portion of a Velcro® pad with adhesive to the bottom of the mold. A lubricious hydrogel formulation is prepared as in Example 1 was the cast on top of the Velcro® in the bottom of the mold in an amount that covered the Velcro® completely and provided 1 to 2 mm of hydrogel liquid above the surface of the fibers. The mold was degassed under vacuum to remove dissolved air and air entrapped in the Velcro fibers. The mold was then covered and placed in an oven at 175° F. for 3 hours to polymerized the hydrogel into a shape-stable solid. The cured lubricious hydrogel was firmly attached to the Velcro fibers by polymerization around and within them.

### Example 10

### Lubricious Hydrogel Pad with Custom Made Adhesive Backing

A custom-made adhesive backing was fabricated from a polyester fusible web tape (Wonder-Web) that was adhered with pressure to one side of a Scotch double-sided adhesive (scrapbooking tape), which had a release liner on one side. This custom-made fibrous sheet with adhesive was adhered to the bottom of a rectangular polypropylene mold, and a lubricious hydrogel formulation as prepared in Example 1 was cast into the mold on top of the fibrous side of the adhesive. The hydrogel was degassed under vacuum and then placed in an oven at 175° F. for 3 hours to polymerize the hydrogel into a shape-stable solid that was firmly adhered to the adhesive. The hydrogel was removed from the mold and its edges were trimmed to match the edges of the adhesive, producing a rectangular acoustic coupling hydrogel pad with one lubricious side for repositionable coupling to skin and one adhesive side for reversible coupling to an ultrasound probe. When tested with an ultrasound probe, the hydrogel with the custom adhesive performed equally as well as both the lubricious hydrogel pad without adhesive and the acoustic coupling gel alone.

### Example 11

### Lubricious Hydrogel Pad for Use in Lithotripsy

The circular dual-layer hydrogel pad or membrane from Example 5 was tested on a lithotripsy machine by placing the tacky side down on the silicone bellows of the machine without any additional lubricant. It was noted that the pad adhered well enough to the bellows surface to prevent it from sliding or being easily repositioned on the bellows. The bellows was then raised to engage with the test chamber and then tested by sending focused shockwaves through it towards a pressure transducer. The dual-layered pad transmitted shockwaves as efficiently as either acoustic coupling gel or a single layer lubricious pad of the invention.

While the present disclosure has been described with reference to preferred embodiments, it should be understood by those familiar with the art that these examples are not limiting, and that a broad array of hydrogel chemistries, compositions, physical forms of the hydrogels and methods of application are contemplated by this invention. The lubricious hydrogels of the present disclosure can also contain many other hydrophilic polymers as slip additives, as well as humectants to retard drying, various salts and emollients, antimicrobial agents to prevent the growth and transmission of pathogenic organisms, and preservative agents to prevent mold growth. The present invention is intended to include modifications that would be apparent to those skilled in the art to which the subject matter pertains without deviating from the spirit and scope of the following claims.

What is claimed:

1. An ultrasound conductive pad comprising:
a first hydrogel membrane comprising:
one or more water-soluble polymers; and
the one or more water-soluble polymers entrapped within a crosslinked hydrogel network;
a first lubricious surface of the first hydrogel membrane; and
a second surface that is less lubricious than the first lubricious surface.

2. The ultrasound conductive pad of claim 1, further comprising a second hydrogel membrane.

3. The ultrasound conductive pad of claim 2, wherein the first hydrogel membrane comprises the first lubricious surface and the second hydrogel membrane comprises the second surface.

4. The ultrasound conductive pad of claim 3, wherein the first hydrogel membrane comprises the one or more water-soluble polymers and the second hydrogel membrane does not include the one or more water-soluble polymers.

5. The ultrasound conductive pad of claim 1, wherein the one or more water-soluble polymers have a molecular weight above 100,000 Daltons.

6. The ultrasound conductive pad of claim 1, wherein the second surface comprises an adhesive surface coupled to the first hydrogel membrane.

7. The ultrasound conductive pad of claim 1, wherein the second surface is configured to reversibly couple with at least one of an ultrasound probe and a lithotripsy bellow.

8. The ultrasound conductive pad of claim 1, wherein the one or more water-soluble polymers comprise at least one of polyvinylpyrrolidone, polyethylene glycol, and polyethylene oxide, and the crosslinked hydrogel network comprises at least one hydrophilic ethylenically-unsaturated monomer and a multi-olefinic crosslinking agent.

9. The ultrasound conductive pad of claim 8, wherein the crosslinked monomer network comprises 2-hydroxyethyl methacrylate and a multifunctional acrylate.

10. The ultrasound conductive pad of claim 9, wherein the first hydrogel membrane comprises between about 0.5 and about 10.0% multifunctional acrylate based on the total weight of 2-hydroxyethyl methacrylate and the multifunctional acrylate.

11. The ultrasound conductive pad of claim 9, wherein multifunctional acrylate comprises at least one of a polyethylene glycol dimethacrylate and polyethylene glycol diacrylate.

12. A method of manufacturing a conductive pad, the method comprising:
preparing a first formulation comprising one or more water-soluble polymers dissolved in water;
preparing a second formulation comprising a water-soluble monomer and a crosslinking agent;
combining the first and the second formulation with a catalyst to form a third formulation;
casting the third formulation into a casting mold; and
curing the third formulation.

13. The method of claim 12, wherein a surface of the casting mold has a water contact angle greater than about 90°.

14. The method of claim 13, further comprising:
forming a first surface at an interface of the surface of the casting mold and the cast third formulation; and
forming a second surface at an interface of the cast third formulation and a gas.

15. The method of claim 14, wherein the first surface has a greater lubricity than the second surface.

16. The method of claim 12, further comprising:
forming a fourth hydrogel formulation not comprising a water-soluble polymer;
casting the fourth hydrogel formulation into the casting mold;
at least partially curing the fourth hydrogel formulation;
casting the third formulation into the casting mold; and
fully curing the third formulation and the fourth hydrogel formulation.

17. The method of claim 12, further comprising applying an adhesive backing to the third formulation.

18. The method of claim 17, wherein the adhesive backing comprises a fibrous material bound to one side of a pressure sensitive adhesive.

19. The method of claim 12, wherein the one or more water-soluble polymers comprise at least one of polyvinylpyrrolidone, polyethylene glycol, and polyethylene oxide, and the water-soluble monomer comprises at least one hydrophilic ethylenically-unsaturated monomer, and the crosslinking agent comprises a multi-olefinic crosslinking agent.

20. The method of claim 12, wherein the water-soluble monomer comprises 2-hydroxyethyl methacrylate and the crosslinking agent comprises a multifunctional acrylate.

21. The method of claim 20, wherein the second formulation comprises between about 0.5% and about 10.0% multifunctional acrylate based on the total weight of 2-hydroxyethyl methacrylate and the multifunctional acrylate.

22. The method of claim 20, wherein multifunctional acrylate comprises at least one of a polyethylene glycol dimethacrylate and a polyethylene glycol diacrylate.
